(19) United States
(12) Patent Application Publication
(10) Pub. No.: US 2012/0220847 A1
(43) Pub. Date: Aug. 30, 2012

(54) ALARM SYSTEMS USING MONITORED PHYSIOLOGICAL DATA AND TREND DIFFERENCE METHODS

(75) Inventors: Victor Skladnev, Vaucluse (AU); Stanislav Tamavskii, Allawah (AU); Thomas McGregor, West Ryde (AU); Nejhdeh Ghevondian, Ryde (AU)

(73) Assignee: AIMEDICS PTY LTD, Eveleigh (AU)

(21) Appl. No.: 13/505,808

(22) PCT Filed: Nov. 4, 2010
(86) PCT No.: PCT/AU2010/001468

§ 371 Date: May 3, 2012

(30) Foreign Application Priority Data
Nov. 4, 2009 (AU) 2009905384

(51) Int. Cl. A61B 5/024; A61B 5/145

(52) U.S. Cl. 600/365

(57) ABSTRACT

A method and system are described for detecting a hypoglycaemic state in a patient. The patient's heart rate is monitored to provide a heart-rate signal. A time-lagged signal is determined as the difference between the heart-rate signal and a time-lagged version of the heart rate-signal. The heart-rate signal is filtered with a low-pass filter to provide a heart-rate trend. An absolute difference between the heart-rate signal and the heart-rate trend is determined to provide an absolute-difference signal. A second time-lagged signal is determined as a difference between the absolute-difference signal and a time-lagged version of the absolute-difference signal. The occurrence of a hypoglycaemic condition is inferred dependent on the time-lagged signal and the second time-lagged signal.

### FIELD OF THE INVENTION

The present invention relates to the design of alarm systems using physiological responses. In particular such systems can be used for the non-invasive monitoring of hypoglycaemia.

### BACKGROUND OF THE INVENTION

Non-invasive monitoring over extended periods using wireless links to interpretation systems provides a potential solution to many significant health medical issues from heart disease detection to aspects of diabetes management.

Diabetes is one of the fastest growing chronic diseases world-wide with an estimated current incidence of over 200 million people. Of this significant and growing population some 10% have type 1 insulin-dependant diabetes mellitus (T1DM) and require regular insulin therapy. Insulin therapy is however associated with a three-fold increased risk of hypoglycaemia (low blood glucose levels). Hypoglycaemia is the most common and feared complication experienced by insulin-dependent patients. Its onset is characterised by symptoms which include sweating, tremor, palpitations, loss of concentration and control. Nocturnal episodes cause particular concern due to the association of extended periods of hypoglycaemia with coma and neurological damage. Detection of hypoglycaemia is problematic due to sampling issues and the relatively wide error bands of consumer devices at low blood-glucose levels.

Current technologies used for diabetes diagnostic testing and self-monitoring are well established. For example, glucose meter manufacturers have modified their instruments to use as little as 2 μl of blood and produce results in under a minute. However, devices which require a blood sample are unsatisfactory in that the sample is painful to obtain, and regular monitoring is not practical, particularly overnight.

U.S. Pat. No. 7,502,644 describes an invasive technique for detecting hypoglycaemia from an analysis of the time interval between ventricular depolarization and repolarisation in conjunction with associated ECG wave shapes and heights.

Minimally invasive continuous glucose monitors have been developed that provide valuable blood glucose data but are limited in their ability to accurately detect the small differences between normal and hypoglycaemia glucose levels.

U.S. Pat. No. 6,882,940 describes a multi-parameter non-invasive approach that seeks to detect hypoglycaemia through the combination of IR spectroscopy and skin temperature/conductivity threshold techniques.

The prior hypoglycaemia detection methods either suffer from being incompatible with the need for continuous monitoring or are insufficiently specific for the detection of this potentially dangerous condition. The fear of hypoglycaemia remains the major limitation to improving diabetic control in patients treated with insulin. There is a need for a convenient and specific hypoglycaemia alarm.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

It is an object of the present invention to overcome, or at least ameliorate, one or more problems of prior art systems.

According to a first aspect of the invention there is provided a method of detecting a hypoglycaemic state in a patient, the method comprising:

monitoring a heart rate of the patient to provide a heart-rate signal;

determining a time-lagged time sequence as the difference between the heart-rate signal and a time-lagged version of the heart-rate signal;

inferring the occurrence of a hypoglycaemic event if the difference exceeds a first specified threshold and

issuing an alarm if the occurrence is inferred.

According to another aspect of the invention there is provided a method of detecting a hypoglycaemic state in a patient, the method comprising:

monitoring a heart rate of the patient to provide a heart-rate signal;

filtering the heart-rate signal with a low-pass filter to provide a heart-rate trend;

determining an absolute difference between the heart-rate signal and the heart-rate trend to provide an absolute-difference time sequence; and

generating a time-lagged signal as a difference between the absolute-difference time sequence and a time-lagged version of the absolute-difference time sequence.

According to a further aspect of the invention there is provided a method of detecting a hypoglycaemic state in a patient, the method comprising:

monitoring a heart rate of the patient to provide a heart-rate signal;

determining a time-lagged signal as the difference between the heart-rate signal and a time-lagged version of the heart rate-signal;

filtering the heart-rate signal with a low-pass filter to provide a heart-rate trend;

determining an absolute difference between the heart-rate signal and the heart-rate trend to provide an absolute-difference signal;

generating a second time-lagged signal as a difference between the absolute-difference signal and a time-lagged version of the absolute-difference signal; and

inferring the occurrence of a hypoglycaemic condition dependent on the time-lagged signal and the second time-lagged signal.

The invention also resides broadly in a system comprising:

a heart-rate monitor for monitoring a heart rate of a patient; and

a processor programmed to detect a hypoglycaemic condition of the patient dependent on trends in the monitored heart rate.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

One or more embodiments of the present invention are described below with reference to the drawings, in which:

FIG. 1A is a schematic diagram of a chest-belt transmitter that may be used in the implementation of the present invention;

FIG. 1B is a schematic diagram of a receiver unit that may be used in conjunction with the transmitter of FIG. 1A;

FIG. 2 is a flow diagram of a method for monitoring a user's heart rate and triggering an alarm if a hypoglycaemia event is detected;

FIG. 3A is an example of an overnight blood glucose measurement;

FIG. 3B shows a heart-rate measurement and a derived low-frequency heart-rate trend corresponding to the glucose measurement of FIG. 3A;

FIG. 3C shows the glucose measurement of FIG. 3A together with an alarm triggered from the trend change and threshold of FIG. 3D;

FIG. 3D is a corresponding graph showing trend changes calculated as a difference between a current trend value at t=1 and an earlier value at t=i−T_{lag} together with a threshold value;

FIG. 4A shows a heart-rate measurement and a trend obtained from a low-pass filter;

FIG. 4B shows an absolute difference between the measurement and trend of FIG. 4A;

FIG. 5 shows an example of a fitted line used to determine a no-alarm window based on an initial blood glucose level measurement; and

FIG. 6 is a flow chart of a method of adjusting parameters of the detection method of FIG. 2 based on additional variables.

### DETAILED DESCRIPTION OF THE EMBODIMENTS

The methods and systems described herein aim to provide solutions to the problem of accurately detecting hypoglycaemia events either as a stand-alone system or in combination with technologies that directly estimate blood glucose levels such as continuous glucose monitors.

The described methods and systems use physiological parameter signatures which in this case distinguish hypoglycaemia. These signatures are derived from time-sequence trend-difference features within frequency ranges and time-windows that are specific to the application, in this case the detection of hypoglycaemia events.

Various embodiments of the system of the present invention have common features. Research by the inventors has shown that regular monitoring of physiological parameters such as an electrocardiogram (ECG) can provide the basis of accurate detection of hypoglycaemia states through establishing whether the difference between the current time-sequence trend and a time-lagged trend in the selected parameter crosses a threshold value. The threshold-crossing time of the selected parameter may be provided to an algorithm which receives other parameter responses and additional information such as a pre-bed-time finger-prick BGL value or otherwise estimated BGL values. An alarm sequence may be activated when a summation algorithm suggests the presence or imminent onset of hypoglycaemia conditions.

The following describes the currently implemented mode of practicing the invention. This description is not intended to limit the general nature of the invention but is given for the purpose of describing a particular embodiment.

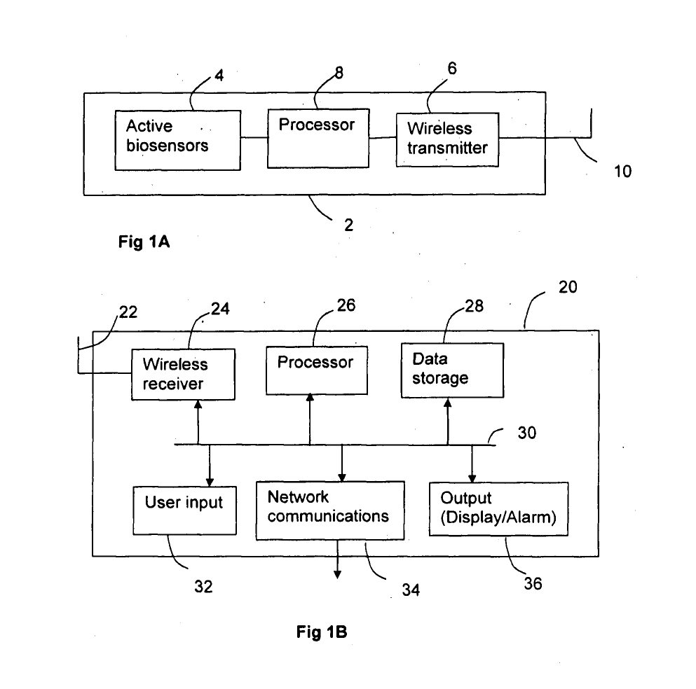

FIGS. 1A and 1B illustrate a system that may be used to implement the methods described herein. In this arrangement, a patient may wear a chest-belt unit 2 which, in use, is located around the patient's the upper thoracic region. The chest-belt unit 2 may have an adjustable elasticated strap which is adapted to engage tightly around the patient's chest using a suitable and a secure fastening system which is relatively easy to engage and disengage to enable the belt unit 2 to be put on and taken off without difficulty. The strap can also be adapted to fit around a child's chest in the same manner as the adult patient. The belt unit 2 incorporates an electronic housing that encloses a wireless transmitter, analogue electronic circuitry and a microcontroller.

As shown in FIG. 1A, the belt unit 2 includes active biosensors 4 that may be skin surface electrodes each adapted to monitor a different physiological parameter. The sensors 4 measure physiological parameters such as skin impedance, ECG and segments thereof, including QT-interval and ST-segment, heart rate and the mean peak frequency of the heart rate. These aspects are further discussed in detail in PCT/AU02/00218, published as WO 02/069798.

The biosensors 4 provide the signals which, after being processed, amplified, and filtered by analogue electronic circuitry, are interfaced to the microcontroller (μC) unit 8. The μC unit 8 digitises the signals using an A/D (analogue-to-digital) converter and provides the digitised signals to a wireless transmitter 6 with an aerial 10.

Associated with the belt unit 2 is a receiver unit 20 which is adapted to process signals monitored by the unit 2 for analysis and alarms. The units 2 and 20 may be encoded to recognise one another for secure communication. As shown in FIG. 1B, the receiver unit 20 has an aerial 22 and wireless receiver 24. Data may be stored in data storage 28 and processed by software running on the processor 26. Data communication between the components of the receiver unit 20 is provided by bus 30. The unit 20 may have one or more output units 36 including a display for displaying information to the user. The outputs 36 may also include an audible alarm.

A network communication interface 34 may also be included. This permits information about the patient's physiological condition to be transmitted elsewhere, for example via an Internet connection to a health-care provider such as an endocrinologist or cardiologist. In another example information may be sent via an SMS messaging service. Thus, for example, if the units 2, 20 are monitoring a child, a message may be sent to the child's parents if an alarm is triggered.

The unit 20 may also include a user input 32 that permits additional information to be entered into the unit 20. For example, if the patient takes a reading of blood glucose level (BGL), this may be entered into the unit 20 using a keypad. Alternatively or additionally, the input 32 may be a data link to other equipment such as a continuous BGL monitor or suitably equipped finger-prick devices.

An example of a suitable monitoring system is the HypoMon described in patent application WO 2004/098405 titled “Patient Monitor”.

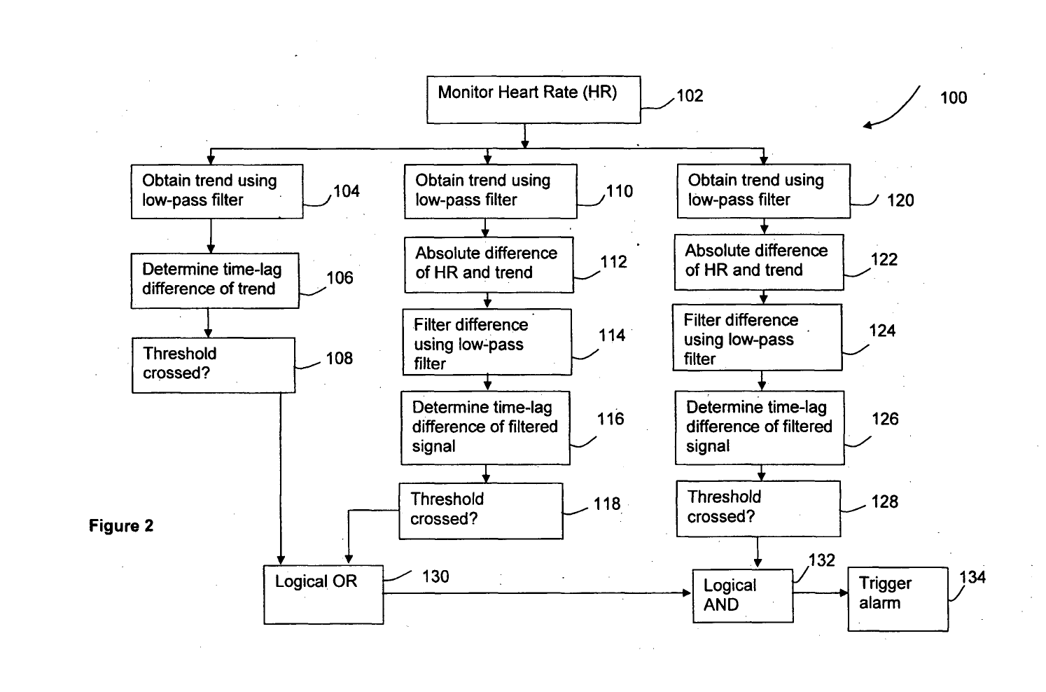

A method 100 for monitoring physiological data to detect a hypoglycaemia event is shown in FIG. 2. A patient's heart rate is monitored (step 102), for example using the units 2, 20 described with reference to FIGS. 1A and 1B. In method 100, the heart rate data is analysed in three different ways (steps 104-108, 110-118 and 120-128 respectively) and the results are combined to trigger an alarm if appropriate. The steps 104-134 may be performed by software running on the processor 26 of the receiver unit 20. It will be appreciated that the method 100 may have different implementations. For example, information may be forwarded from the unit 20 to a remote server for processing. The method 100 could also be performed in a distributed fashion, where different portions of the method are carried out using different processors. The method 100, or parts of the method 100, may also be performed using other processing means such as analog circuitry, application-specific integrated circuits (ASICs) or field-programmable gate arrays (FPGAs).

Time-Lag Trend

In step 104 the patient's heart rate is passed through a low-pass filter to obtain a low-frequency heart-rate trend. In one arrangement the filter has a time constant of 1.6 hours. Methods of selecting parameter values for the method 100 will be discussed below. The filters may be implemented as multi-stage RC filters or similar. The filters may also be implemented as digital filters, for example as software running on processor 8 or 26.

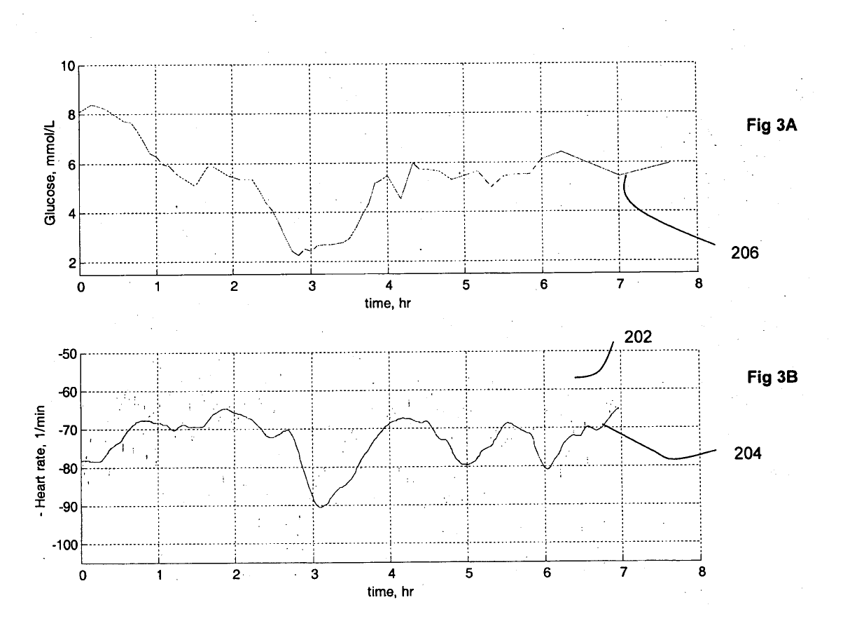

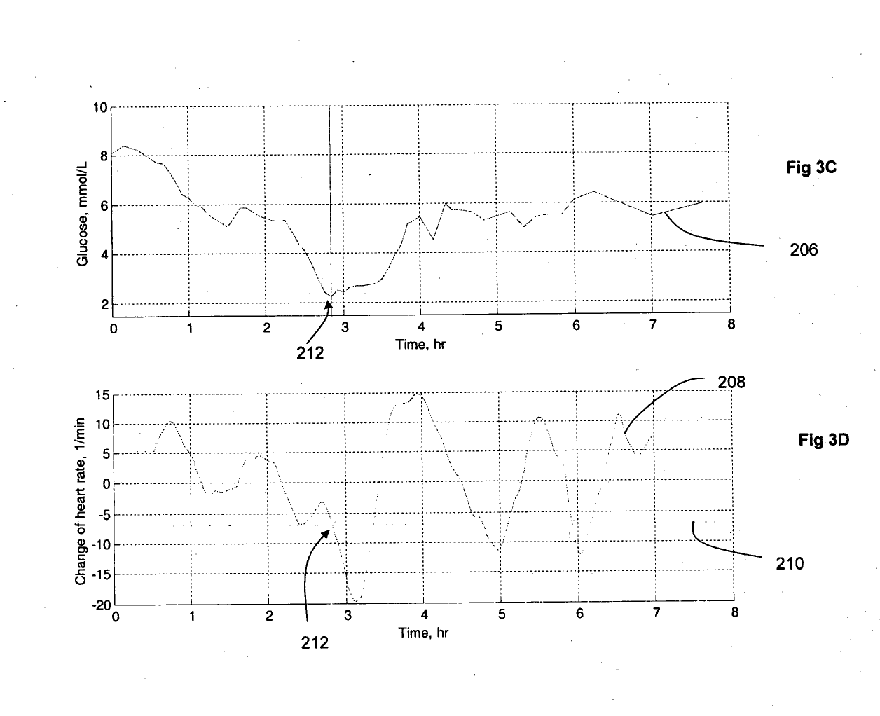

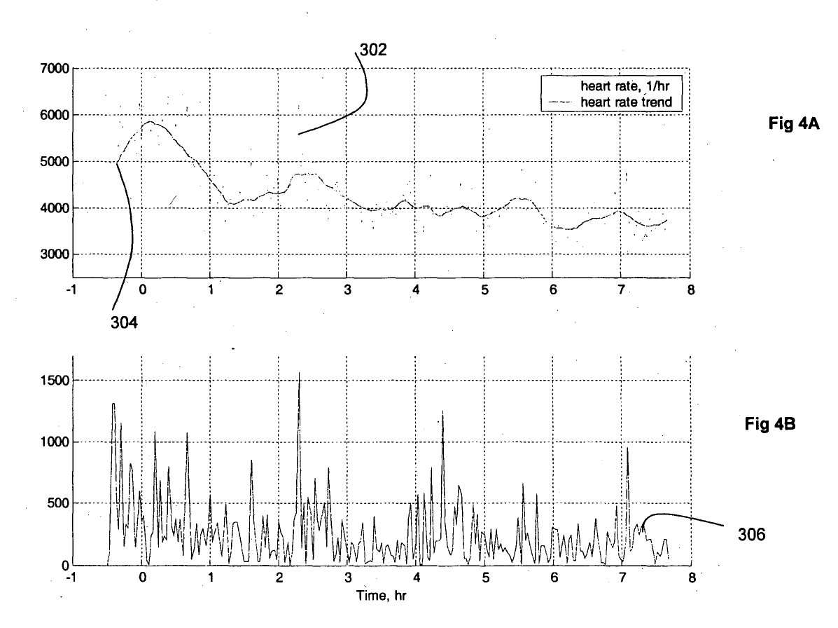

The method 100 is illustrated with the trends shown in FIGS. 3 and 4, which were derived from a T1DM sufferer. FIG. 3A shows an overnight profile of the patient's blood glucose level 206. FIG. 3B shows the patient's raw heart rate trend 202 over the same time period. Line 204 is a low-frequency heart-rate trend output from a low pass filter (in this case with a filtering time of around 0.5 hour). Trend 204 is delayed with respect to the raw data 202 as an inherent effect of the filter.

In step 106 a time-lag trend is determined as a difference between a value of the trend 204 at time t=i and a past value of the trend 204 at time t=(i−T_{lag}). In the inventors' view step 106 is a normalizing process that establishes a dynamic baseline for the process before the occurrence of hypoglycaemia. The time-lag trend monitors the change in heart rate with respect to the dynamic baseline.

Line 208, shown in FIG. 3D, is the time-lag trend for the specific example. Here, T_{lag} is 0.5 hour. In another arrangement a lag value of 1.6 hours has been used.

In step 108 the monitoring software checks whether a specified threshold has been crossed. In the example of FIG. 3D line 210 designates the relevant threshold. Point 212 shows where the time-lag trend 208 crosses the threshold 210. FIG. 3C illustrates how the threshold crossing maps onto the patient's blood glucose level 206. The triggering event corresponds to a drop in the patient's BGL.

Difference Between Heart-Rate and Heart-Rate Trend

Steps 110-118 represent another analysis of the input heart rate. In step 110 the heart rate is filtered using a low-pass filter to provide a low-frequency trend. In one implementation the time constant of the filter is 0.3 hours. Then, in step 112, the absolute difference between the raw heart-rate data and the low-frequency trend is determined. A delayed version of the raw data may be used when determining the absolute difference. The delay is selected to match the delay inherent in the low-pass filtering.

Steps 110 and 112 are illustrated in FIGS. 4A and 4B. Line 302 is raw heart-rate data and line 304 is the filtered low-frequency trend. Line 306 is the absolute difference between lines 302 and 304.

The absolute difference signal is then processed in a similar way to the method of steps 104-108. That is, steps 114, 116 and 118 correspond to steps 104, 106 and 108, although the parameters used in processing may differ.

In step 114 the absolute difference signal is passed through a low-pass filter to obtain a low-frequency difference trend. In one arrangement the filter has a time constant of 2.1 hours.

In step 116 a time-lag trend is determined as a difference between a value of the low-frequency difference trend at time t=i and a past value of the trend at time t=(i=(i−T_{lag}). The time T_{lag} need not be the same as the lag time used in step 106. In one arrangement the T_{lag} for step 116 is 2.1 hours. Then, in step 118, the monitoring software checks whether the output signal from step 116 crosses a specified threshold. If so, an intermediate flag is triggered.

Steps 120-128 represent a third strand of processing of the heart rate signal. Steps 120-128 correspond to the steps 110-118 but use a different frequency pass-band. The processing of steps 120-128 takes into account higher-frequency information than is considered in the processing of steps 110-118.

In step 120 the heart rate is filtered using a low-pass filter to provide a low-frequency trend. In one implementation the time constant of the filter is 0.3 hours. Then, in step 122, the absolute difference between the raw heart-rate data and the low-frequency trend is determined. A delayed version of the raw data may be used when determining the absolute difference. The delay is selected to match the delay inherent in the low-pass filtering.

Steps 120 and 122 may in fact be the same as steps 110 and 112. That is, if the low-pass filter of step 110 is the same as the filter used in step 110 there is no need for separate steps 120, 122 and the output of step 112 may serve as the input to steps 114 and 124.

In step 124 the absolute difference signal is passed through a low-pass filter to obtain a second low-frequency difference trend. In one arrangement the filter has a time constant of 0.17 hours. Consequently, the difference trend output from step 124 includes higher-frequency information than the difference trend output from step 114.

In step 126 a time-lag trend is determined as a difference between a value of the second low-frequency difference trend at time t=i and a past value of the trend at time t=(i−T_{lag}). The time T_{lag} need not be the same as the lag time used in step 106 or 116. In one arrangement the T_{lag} for step 126 is 0.17 hours. That is, the time lag signal output from step 126 relates to higher-frequency information than is represented in the output of step 116.

Then, in step 128, the monitoring software checks whether the output signal from step 126 crosses a specified threshold. If so, an intermediate flag is triggered.

The thresholds used in steps 108, 118 and 128 may differ from one another.

The alarm method 100 combines the outputs of steps 108, 118 and 128. Step 130 is a logical OR operation. If step 108 detects a threshold crossing OR step 118 detects a threshold crossing, then the logical OR of step 130 triggers a further intermediate flag, which is provided to the logical AND function of step 132. The other input to the logical AND is the output of step 128. If the OR function 130 is triggered AND step 128 detects a threshold crossing within a specified time window (for example 1.2 hours), then in step 134 an alarm is triggered by the receiver unit 20. For example, an audible alarm may be sounded, or a message may be transmitted to a carer.

Test results obtained by the inventors suggest that method 100 provides an alarm for overnight hypoglycaemia events based on heart rate trend differences with an algorithm structure having inter-subject stability.

The structure of method 100 may be summarized as follows:

α(alarm)=β[[T(a) OR T(b)] AND Ψ[T(c)]] AND T (w)

Where: T (a) is the response time of the time-lagged difference of the low pass filter components of heart rate (low pass filter time constant 1.6 hours and lag 1.6 hours);

T (b) is the response time of the absolute difference between heart rate and heart rate trend with a 0.3 hour time constant which is further converted to a trend difference as in T (a) where the filter time constant is 2.1 hours and the lag is 2.1 hours;

T (c) varies from T (b) in that the final low-pass filter has a time constant of 0.17 hours and a lag of 0.17 hours. Additionally the time window for the associated AND function is 1.2 hours.

T (w) is a time window derived from initial conditions such as pre-bed time finger-prick BGL.

Time Window

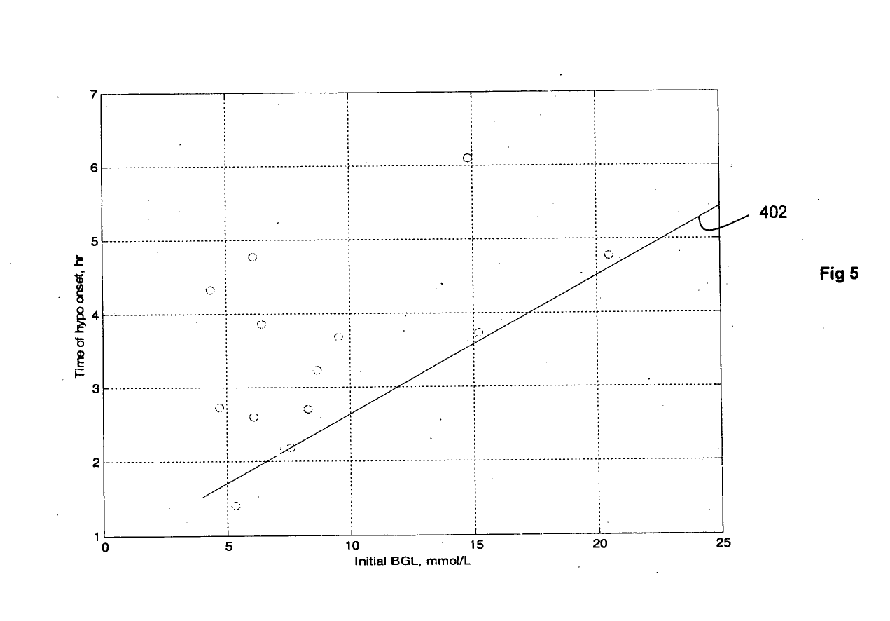

The time window T(w) is based on the observation that patients having higher blood glucose levels at the beginning of the night tend to experience hypoglycaemia later in the night than patients with relatively low initial BGL. This is illustrated in FIG. 5, which shows lapsed time to the onset of hypoglycaemia versus the patients' initial BGL. Line 402 is an example of the no-alarm time window vs the initial BGL. This observation has been used to reduce the number of false alarms by disregarding alarms that are triggered in the area below line 402. To implement this window T(w), a measurement of the patient's BGL is made at the beginning of the night, for example using a finger-prick measurement. The measurement may be keyed into unit 20 using the user input 32. The monitoring software running on unit 20 takes the BGL measurement into account and disregards alarms triggered in step 134 in the initial time window.

Selecting Parameter Values

The method 100 includes several parameters, including time-constants for the low pass filters, lag times for calculating the lagged signals and the values of the thresholds used in steps 108, 118 and 128. These parameters may be set by accumulating patient data including information about the onset of hypoglycaemia, and dividing the data into training data sets and test data sets. The parameter values may be determined by training algorithms that optimize the values based on the training sets. The optimized parameter values may be tested on the test data sets. Such procedures may serve to increase the detection accuracy of the method and to reduce the number of false alarms.

One method for identifying stable signatures within the complex system nature of T1DM sufferer's response to hypoglycaemia was as follows. Selected non-invasive physiological parameters along with regular venous BGL readings on gold standard (YSI) devices were monitored on 130 T1DM volunteers over a range of day/night hypoglycaemic clamp and natural conditions. Analysis of this data was guided by the hypothesis that hypoglycaemia events stimulate physiological responses which show frequency, time-lag and time-window features that have inter-subject stability. Stability evaluations on potential features were then carried out in an iterative manner by segregating the data into training and evaluation data sets. The stability of the discovered signatures was then confirmed in a blinded prospective overnight trial on 52 previously unseen T1DM sufferers.

Using Dynamic Parameter Settings

The alarm thresholds and parameters such as decision integration times used in the described methods can be fixed or dynamic depending on the nature of the additional information available. For example, direct estimates of blood glucose levels (BGL) and trends from a continuous glucose monitor may be integrated into the alarm system in the form of a logic tree of the following general form:

a) At high BGL estimates, ignore all alarms over a specified time window;

b) At near-normal BGL estimates, raise the threshold of alarm features;

c) At low BGL estimates or in the event of significant trends to low BGLs, lower the alarm thresholds for selected features; and

d) At very low BGL estimates activate the alarm.

In this manner allowances may be made for variations in estimation accuracy over BGL ranges.

Alternatively, instead of adjusting the thresholds, scaling factors may be used to take additional information into account. For example, with reference to FIG. 2, a scaling factor may be applied to one or more of the trends before checking whether the trends have crossed the .specified threshold (e.g. in steps 108, 118 and 128). Thus, a scaling factor may be used as a multiplier for the time-lag difference obtained in step 106, and/or the time lag difference determined in step 116 and/or the time-lag difference obtained in step 126.

For example, direct estimates of blood glucose levels (BGL) and trends from a continuous glucose monitor may be integrated into the alarm system in the form of a logic tree of the following general form:

a) At high BGL estimates, ignore all alarms over a specified time window;

b) At near-normal BGL estimates, reduce one or more of the scaling factors to reduce the probability of the scaled trend exceeding the specified threshold;

c) At low BGL estimates or in the event of significant trends to low BGLs, increase one or more of the scaling factors to increase the probability of the scaled trend exceeding a specified threshold; and

d) At very low BGL estimates activate the alarm.

In this manner allowances may be made for variations in estimation accuracy over BGL ranges. The scaling coefficients may be varied dependent on the BGL value at the beginning of the night or on the history of BGL from the beginning of the night through to the latest reading.

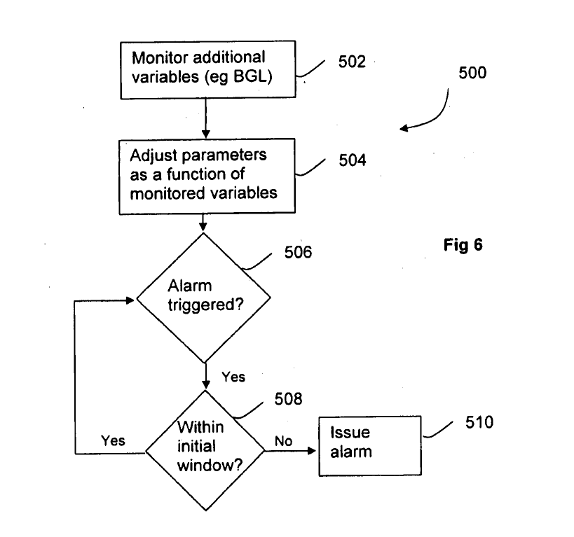

This is further illustrated in method 500 (see FIG. 6). In step 502, additional variables such as BGL are monitored, in addition to the heart rate monitoring of step 102. Then, in step 504, one or more parameters of the alarm method 202 are adjusted, for example as described in the foregoing paragraph. These adjustments, may be performed by software running on the receiver unit 20. Other arrangements may be used. For example, the adjustments may be determined by software running on a remote server and transferred to the relevant data registers 28 of the receiver unit 20.

In step 506 the alarm method 100 runs. If the method triggers an alarm (the YES option of step 506), then in step 508 the monitoring software checks whether the alarm should be ignored because it has been triggered within a specified time window. If appropriate, the alarm is issued in step 510, otherwise process flow returns to step 506 to continue monitoring the patient.

It will be evident to those experienced in device algorithm development that some details of the methods described above are illustrative of structure rather than form as specific device features will substantially influence the optimum solutions.

The foregoing describes only some embodiments of the present invention, the embodiments being illustrative and not restrictive. The intended application of the alarm system will determine the structure of the basic alarm algorithm.

Although this specification concentrates on a system and method for the detection of hypoglycaemia, it should be understood that the invention has wider application.

It will be understood that the invention disclosed and defined in this specification extends to all alternative combinations of two or more individual features mentioned or evident from the text or drawings. All of these different combinations constitute various alternative aspects of the invention.

In the context of this specification, the word “comprising” or its grammatical variants is equivalent to the term “including” and should not be taken as excluding the presence of other elements or features.

1. method of detecting a hypoglycaemic state in a patient, the method comprising:
monitoring a heart rate of the patient to provide a heart-rate signal;
determining a time-lagged time sequence as the difference between the heart-rate signal and a time-lagged version of the heart-rate signal;
inferring the occurrence of a hypoglycaemic event if the difference exceeds a first specified threshold and
issuing an alarm if the occurrence is inferred.

2. The method of claim 1 comprising filtering the heart-rate signal with a low-pass filter to provide a heart-rate trend, wherein the time-lagged time sequence is determined as the difference between the heart rate trend and a time-lagged version of the heart-rate trend.

3. The method of claim 1 comprising varying the first specified threshold dependent on one or more measured patient parameters.

4. The method of claim 3 wherein the patient parameter comprises a measured blood glucose level.

5. The method of claim 4 wherein, if the measured blood glucose level is high, the first threshold is adjusted to reduce the likelihood of inferring the occurrence of a hypoglycaemic event.

6. The method of claim 4 wherein, if the measured blood glucose level is near normal values or at low levels, the first threshold is adjusted to increase the likelihood of inferring the occurrence of a hypoglycaemic event.

7. The method of claim 1 wherein the method of detecting a hypoglycaemic state is commenced at a start time and the method comprises:
determining a no-alarm window period dependent on a blood glucose value of the patient associated with the start time, wherein no alarm is issued if a hypoglycaemic event is inferred between the start time and an end time of the no-alarm window period.

8. The method of claim 7 wherein the duration of the no-alarm window is increased for higher levels of blood glucose associated with the start time.

9. A method of detecting a hypoglycaemic state in a patient, the method. comprising:
monitoring, a heart rate of the patient to provide a heart-rate signal;
filtering the heart-rate signal with a low-pass filter to provide a heart-rate trend;
determining an absolute difference between the heart-rate signal and the heart-rate trend to provide an absolute-difference time sequence; and
generating a time-lagged signal as a difference between the absolute-difference time sequence and a time-lagged version of the absolute-difference time sequence.

10. The method of claim 9 comprising inferring the occurrence of a hypoglycaemic condition if the time-lagged signal exceeds a specified threshold.

11. A method of detecting a hypoglycaemic state in a patient, the method comprising:
monitoring a heart rate of the patient to provide a heart-rate signal;
determining a time-lagged signal as the difference between the heart-rate signal and a time-lagged version of the heart rate-signal;
filtering the heart-rate signal with a low-pass filter to provide a heart-rate trend;
determining an absolute difference between the heart-rate signal and the heart-rate trend to provide an absolute-difference signal;
generating a second time-lagged signal as a difference between the absolute-difference signal and a time-lagged version of the absolute-difference signal; and
inferring the occurrence of a hypoglycaemic condition dependent on the time-lagged signal and the second time-lagged signal.

12. The method of claim 11 wherein the occurrence of the hypoglycaemic condition is inferred if the time-lagged signal crosses a first specified threshold and the second time-lagged signal crosses a second specified threshold.

13. The method of claim 12 comprising varying the first specified threshold and/or the second specified threshold dependent on one or more measured patient parameters.

14. The method of claim 12 comprising multiplying the time-lagged signal and/or the second time-lagged signal by a scaling factor dependent on one or more measured patient parameters.

15. The method of claim 13 wherein the patient parameter comprises a measured blood glucose level.

16. The method of claim 15 wherein, if the measured blood glucose level is high, the first threshold and/or the second threshold are adjusted to reduce the likelihood of inferring the occurrence of a hypoglycaemic event.

17. The method of claim 15 wherein, if the measured blood glucose level is near normal values or at low levels, the thresholds are adjusted to increase the likelihood of inferring the occurrence of a hypoglycaemic event.

18. The method of claim 11 wherein the method of detecting a hypoglycaemic state is commenced at a start time and the method comprises:
determining a no-alarm window period dependent on a blood glucose value of the patient associated with the start time, wherein no alarm is issued if a hypoglycaemic event is inferred between the start time and an end time of the no-alarm window period.

19. A system for detecting a hypoglycaemic state in a patient, comprising:
a heart-rate monitor for monitoring a heart rate of the patient; and
a processor in data communication with the heart-rate monitor, programmed to detect a hypoglycaemic condition of the patient using the method of claim 1.

20. A computer program product comprising machine-readable program code recorded on a machine readable recording medium, for controlling the operation of a data processing apparatus on which the program code executes to perform a method of detecting a hypoglycaemic condition of the patient using the method of claim 1.

21. (canceled)
